(19) United States
(12) Patent Application Publication
(10) Pub. No.: US 2026/0014224 A1
(43) Pub. Date: Jan. 15, 2026

(54) CROCUS SATIVUS FLOWER EXTRACTS, COMPOSITIONS COMPRISING THEM AND THEIR USE IN ORAL CARE

(71) Applicant: ISP INVESTMENTS LLC, Wilmington, DE (US)

(72) Inventors: Elodie OGER, Antibes (FR); Imane GARCIA, Nice (FR); Christophe CAPALLERE, Nice (FR); Sandrine PINACOLO, SAINT VALLIER DE THIEY (FR); Laura MOURET, CAGNES SUR MER (FR)

(73) Assignee: ISP INVESTMENTS LLC, Wilmington, DE (US)

(21) Appl. No.: 18/856,718

(22) PCT Filed: Apr. 4, 2023
(86) PCT No.: PCT/EP2023/058789

§ 371 Date: Oct. 14, 2024

(30) Foreign Application Priority Data
Apr. 14, 2022 (FR) 2203447

(51) Int. Cl. A61K 36/88; A61K 8/9794; A61P 1/02; A61P 31/04; A61Q 11/00

(52) U.S. Cl. CPC A61K 36/88; A61K 8/9794; A61P 1/02; A61P 31/04; A61Q 11/00; A61K 2236/333; A61K 2236/53; A61K 2800/782

(57) ABSTRACT

The present invention relates to compositions for oral hygiene comprising Crocus sativus flower extracts and their uses for protecting the oral cavity. The invention also relates to therapeutic compositions for use in the prevention and treatment of diseases of the oral cavity, in particular periodontitis and gingivitis.

### TECHNICAL FIELD

The present invention relates to the technical field of oral hygiene and the therapeutic treatment of oral pathologies. It relates more particularly to compositions comprising Crocus sativus flower extracts for oral hygiene or protection of the oral cavity. The invention also relates to compositions comprising Crocus sativus flower extracts for their therapeutic uses in the prevention and treatment of disorders of the oral cavity, in particular bleeding, inflammation, tooth loosening or loss of conjunctival substance associated with periodontitis and gingivitis caused by the Porphyromonas gingivalis bacterium.

### TECHNICAL BACKGROUND TO THE INVENTION

The oral cavity is colonised by a large number of micro-organisms that make up the oral microbiome. In a healthy oral cavity, the micro-organisms are fundamentally commensal bacteria that organise themselves to maintain the balance of this ecosystem, although this does not rule out the presence of pathogenic bacteria.

The oral cavity is frequently the site of diseases such as gingivitis and periodontitis. Gingivitis is a reversible inflammatory condition, generally caused by an accumulation of bacterial biofilm, which manifests itself as gingival redness, oedema and sometimes bleeding, but without tooth loosening. If gingivitis is not treated in time, the inflammation can become chronic and develop into periodontitis, characterised by resorption of the alveolar bone, destruction of the periodontal ligament and, ultimately, loosening of the teeth.

One of the major causes of periodontitis is an imbalance in the oral microbiome, combined with a weak immune response. Certain pathogenic bacteria then combine to supplant commensal bacteria. This association of bacteria has been described as the “red complex” and consists of the species Porphyromonas gingivalis, Tannerella forsythia and Treponema denticola (Socransky S S, Haffajee A D, Cugini M A, Smith C, Kent R L Jr. in J Clin Periodontol. 1998; 25 (2): 134-144).

Porphyromonas gingivalis plays a prominent role in biofilm formation (Hajishengallis G, Darveau R P, Curtis M A., Nat Rev Microbiol. 2012; 10 (10): 717-725) and in the immune system's avoidance strategy (Hajishengallis G., J Oral Biosci. 2011; 53 (3): 233-240).

Porphyromonas gingivalis secretes an enzyme called gingipain, which enables the bacterium to capture the iron contained in the haem of host tissues. This enzyme is also involved in breaking down the cell junctions of the gingival epithelium, inducing inflammation, gum oedema, a tendency to bleed and destruction of the type I and III collagen fibres of the gingival connective tissue.

The problem that the invention sets out to solve is to provide a new active ingredient capable of targeting the activity of the enzyme gingipain, in order to specifically limit the pathogenic activity of the Porphyromonas gingivalis bacterium while preserving the oral microbiome and limiting the consequences of periodontitis and gingivitis.

The inventors discovered that a Crocus sativus flower extract had an inhibitory effect on the enzymatic activity of gingipain and could effectively limit the development of Porphyromonas gingivalis bacteria associated with periodontitis and gingivitis.

Crocus sativus is a herbaceous plant of the Iridaceae family cultivated mainly for the pistil of its flowers called saffron, used in food as a spice or for its antioxidant and anti-inflammatory properties, particularly in oral or dental diseases (Forouzanfar A. et al., Iran J Med Sci. 2016 May; 41).

Most of the extracts described in the prior art are extracts of pistils or whole flowers, which therefore include the pistil. These extracts have the disadvantage of containing a high quantity of compounds such as safranal, a substance classified as a skin irritant, eye irritant and skin sensitiser under the Globally Harmonised System of Classification and Labelling of Chemicals (UN-GHS).

Patent documents CN11330420A, CN102657747A and CN109646535A describe extracts obtained from flowers without pistils using absolute ethanol or hydroethanol solvents.

Patent FR2928835B1 describes the preparation of an extract of Crocus sativus petals obtained by maceration for several days in 70% ethanol. The extract obtained contains approximately 50% flavonoid-type polyphenols, including a large quantity of kaempferol, which gives it skin depigmenting properties.

Hydroalcoholic solvents with a high alcohol content such as those used in patent FR2928835B1 are also likely to extract allergenic compounds such as geraniol or linalool because these molecules have a high affinity for this type of solvent.

The present invention describes the use of a mainly aqueous hydroalcoholic solvent. This reduces the extraction of these undesirable compounds. The inventors have shown that the extract of the invention contains none of the 57 substances identified as potential cosmetic allergens by the Scientific Committee on Consumer Safety. In addition, the extraction yield of polar phytocompounds, such as proteins, amino acids or phenolic acids, is improved compared to hydroethanol extraction.

Another problem which the invention sets out to solve is to provide a new aqueous extract of Crocus sativus prepared from whole flowers without pistils and not comprising allergenic, irritant or potentially toxic molecules, for use in oral hygiene or the prevention and treatment of oral pathologies.

The use of a mainly aqueous hydroalcoholic solvent also has the advantage of increasing the extraction yield of phytocompounds of interest, such as sugars, amino acids, proteins, vitamins and organic acids, which are not found, or only to a limited extent, in the extracts of the prior art.

The raw material of the invention comprises only the pistil-free flowers of Crocus sativus, meeting the demand for naturalness in today's oral care and hygiene market while offering remarkable biological efficacy.

### SUMMARY OF THE INVENTION

The inventors have developed a new Crocus sativus extract comprising from 9 to 13 g/Kg of dry matter, from 5.5 to 8.5 g/kg of total sugars, from 2.5 to 5.5 g/kg of proteins, from 0.35 to 0.55 g/Kg of amino acids, from 0.6 to 1.2 g/kg organic acids, 0.4 to 12 g/kg total phenolic compounds including 150 to 450 mg/kg kaempferol sophoroside, 5 to 30 mg/kg kaempferol glucoside and 10 to 40 mg/kg quercetin diglucoside.

In a particular embodiment, the above extract is obtained by maceration of flowers devoid of pistils in a predominantly aqueous hydro-polyalcoholic solvent comprising from 20 to 50% alcohol by weight relative to the total weight of solvent, it being possible for the alcohol to be chosen from propanediol, propylene glycol, butylene glycol and pentanediol.

The invention secondly relates to an oral hygiene or therapeutic composition comprising, as active ingredient, an effective amount of Crocus sativus extract, and a physiologically acceptable medium.

The invention thirdly relates to the non-therapeutic use of the composition for oral hygiene.

The invention fourthly relates to a composition comprising a Crocus sativus flower extract for therapeutic use in the prevention or treatment of diseases of the oral cavity, in particular gingivitis and/or periodontitis.

### BRIEF DESCRIPTION OF THE DRAWINGS

The invention and the advantages arising therefrom will be better understood on reading the description and the non-limiting embodiments which follow, illustrated with reference to the annexed figures in which:

FIG. 1 Shows modulation of arginine-specific gingipain activity by a 1% Crocus sativus extract.

FIG. 2 Shows modulation of lysine-specific gingipain activity by a 1% Crocus sativus extract.

FIG. 3 Shows a test for inhibition of haemolysis induced by Porphyromonas gingivalis bacteria in the presence of a 1% Crocus sativus extract.

FIG. 4 Shows measurement of the expression of CD14+ and CD163 markers on the surface of type 2 macrophages, treated or not with 1% Crocus sativus extract.

FIG. 5 Shows measurement of the mean relative fluorescence of CD14 and CD163 markers on the surface of type 2 macrophages, treated or not with 1% Crocus sativus extract.

### DETAILED DESCRIPTION OF THE INVENTION

### Definitions

All terms used in this description have the most widely known meaning unless otherwise stated. For the purposes of the invention, the following terms are defined as follows:

“Oral cavity” means the area bounded by the teeth, tongue, hard palate and soft palate and substantially covered by a mucous membrane. A distinction is made between the masticatory mucosa (gum and hard palate), the covering mucosa (labial, palatal, vestibular, floor of the mouth and lateral surfaces of the tongue), while the mucosa of the lips and that of the dorsal surface of the tongue make up the specialised mucosa.

“Oral hygiene” means non-therapeutic preventive or protective care for the healthy oral cavity.

“Crocus sativus flower” means the entire flower consisting of the petals and stamens, but without the pistils.

“Hydro-polyalcoholic” means a mixture of at least one polyol and water, the latter comprising at least two alcohol radicals, more precisely from 2 to 9 alcohol radicals and from 3 to 12 carbon atoms.

“Organic acids” means derivatives of the catabolism of amino acids, fatty acids and sugars containing an acid function. This group includes alpha-hydroxylated (AHA) or polyhydroxylated carboxylic acids such as lactic, malic and citric acids, carboxylic acids derived from fruit sugars or any other plant parts such as uronic acids, and diacids such as succinic acid.

“Phenolic compounds” or “polyphenols” means molecules of plant origin which have an aromatic ring bearing one or more hydroxyl groups, such as phenolic acids, flavonoids or their derivatives, tannins and all other polyphenols.

“Flavonoids” means phenolic compounds all sharing the same basic structure formed by two aromatic rings linked by three carbons.

“Glycosylated flavonoids” refers to flavonoids linked to one or more sugars, such as kaempferol sophoroside, kaempferol glucoside and quercetin diglucoside.

“Phytocompounds of interest” refers to all the molecules present in the Crocus sativus flower extract of the invention, in particular sugars, phenolic compounds, proteins, amino acids, vitamins and organic acids, which are potentially responsible for the beneficial biological effects observed.

When a range of values is described, the bounds of this range must be understood as explicitly including all the intermediate values of the range. For example, a value range between 1% and 10% should be understood to include 1%, 2%, 3%, 4%, 5%, 6%, 7%, 8%, 9%, and 10%, as well as all decimal values between 1% and 10%.

Numerical percentage values are percentages by weight, i.e. the weight of a compound relative to the total weight of the intended mixture, unless otherwise specified.

The compositions described in this application may “comprise”, “consist of” or “consist substantially of” essential compounds or optional ingredients.

“Consist substantially of” means that the composition or component may include additional ingredients, if these additional ingredients do not modify the basic characteristics or novel characteristics of the composition or use described in this application.

A “physiologically acceptable medium” means a vehicle suitable for mucosal contact, without toxicity, irritation, undue allergic response or similar or intolerance reaction, and proportionate to a reasonable benefit/risk ratio.

“Topical application” means applying the composition comprising the Crocus sativus flower extracts according to the invention to the surface of a mucosa.

The term “anti-haemolytic” refers to a compound that has the property of preventing the destruction of red blood cells by the bacterium Porphyromonas gingivalis.

“Orientation of the inflammatory response” means stimulating a particular category of the macrophage population in order to obtain an anti-inflammatory or pro-inflammatory response.

“Effective quantity” refers to the minimum quantity of extract according to the invention necessary to obtain at least one of the biological activities sought, in particular to reduce the enzymatic activity of gingipains, to reduce haemolysis, to exhibit anti-inflammatory activity or any other biological marker studied, without this quantity being toxic.

It is understood that the invention concerns mammals and more particularly human beings.

Crocus sativus Flower Extraction Process

The invention firstly relates to an extract of flowers of the species Crocus sativus, obtained by the following process:

In step a), the fresh or dried flowers without pistils are placed in the presence of a predominantly aqueous hydro-polyalcoholic solvent comprising from 20 to 50% alcohol by weight relative to the total weight of the solvent.

For the purposes of the invention, the hydro-polyalcoholic solvent is therefore a mixture of between 20 and 50% (w/w) polyalcohol in water.

The Crocus sativus flowers used are pistil-less, meaning that the three red stigmas of the pistil that make up saffron have been removed by hand.

Dried Crocus sativus flowers are preferred.

Preferably, the flowers are crushed before the hydro-polyalcoholic solvent is added, in order to optimise exchanges between the extractive solution and the plant material.

Preferably, the ratio of plant matter to extraction solution is between 0.5 and 20% w/w, more preferably between 2 and 10% and even more preferably this ratio is 2.5%.

The hydro-polyalcoholic solvents used have a solubility parameter and a dynamic viscosity three times higher than mono-alcoholic solvents. The extraction of more complex molecules such as proteins, polysaccharides and polyphenols is therefore favoured in comparison with a mono-alcoholic solvent, while also allowing small molecules such as sugars and organic acids to be drawn into the extraction process.

Compared with an extraction carried out in pure water, the extraction yield according to the process of the invention is improved and is around 40%, which is a yield that is difficult to achieve using maceration extraction techniques.

In an advantageous embodiment, the polyalcohol is chosen from propanediol, propylene glycol, butylene glycol or pentanediol, or any mixture of these solvents.

In a preferred embodiment, the hydro-polyalcoholic solvent is propanediol or butylene glycol at a ratio of 25% to 35% alcohol.

In another preferred embodiment, the hydro-polyalcoholic solvent preferably comprises 30% alcohol.

In another preferred embodiment, the hydro-polyalcoholic solvent is propanediol at a ratio of 30%.

The presence of propanediol in the extraction solvent also has the advantage of stabilising the phytocompounds in the final extract throughout the extraction process and helps to protect the extract from contamination by micro-organisms.

In step b), the mixture is gently stirred for 30 minutes to two hours at a temperature of between room temperature and 80° C.

Advantageously, the moderate agitation is of the stirring type.

The temperature is adjusted and maintained preferably between 20 and 60° C., more preferably between 3° and 50° C., even more preferably at 45° C.

Advantageously, the maceration time is between 30 minutes and 1.5 hours and even more preferably the maceration time is 1 hour.

At the end of step b) the pH is checked and must be between 4.0 and 7.0.

In step c), the mixture obtained in b) is purified to eliminate the solid vegetable matter and recover the liquid part.

This purification step can be carried out by any method known to a person skilled in the art, but preferably the purification is carried out by centrifugation and in particular by centrifugation at 4000 g for 10 min.

In step d), the extract obtained in the previous step is clarified by at least six sequential filtration steps using filters with decreasing porosities ranging from 30 to 0.2 μm.

In step e), sterilising filtration is carried out on a sterile 0.22 μm porosity filter. The pH is measured but not adjusted, and should be between 4.5 and 6.5.

Crocus sativus Flower Extract

The extract obtained by the process described above is analysed and has the following characteristics, it has an amber to dark amber colour, the extract has a slightly viscous consistency and a slightly opalescent to opalescent appearance. The density is 1.01 to 1.04 g/cm³, the pH is between 4.5 and 6.5. This extract contains from 9 to 13 g/Kg of dry matter, and includes from 5.5 to 8.5 g/kg of total sugars, from 2.5 to 5.5 g/kg of proteins, from 0.35 to 0.55 g/Kg of amino acids, from 0.6 to 1.2 g/kg of organic acids, 0.4 to 1.2 g/kg of total phenolic compounds, including 150 to 450 mg/kg of kaempferol sophoroside, 5 to 30 mg/kg of kaempferol glucoside and 10 to 40 mg/kg of quercetin diglucoside.

The extract obtained therefore contains between 0.17 g/kg and 0.55 g/kg of flavonoid-type polyphenols.

The extract obtained also contains 1.2 to 3.4 mg/kg of B vitamins, including 0.5 to 1.5 mg/kg of vitamin B5.

The extract thus obtained may optionally be diluted in a physiologically acceptable solvent for therapeutic use. Examples of physiologically acceptable solvents include, but are not limited to, water, glycerol, ethanol, propanediol and its natural version derived from corn, butylene glycol, dipropylene glycol, ethoxylated or propoxylated diglycols, cyclic polyols or any mixture of these solvents. The addition of preservatives may also be considered to ensure optimum preservation of the extract over time. Glycerine combined with all types of water-soluble preservatives such as sodium benzoate or potassium sorbate at a final concentration of 0.5%, or phenoxyethanol at a final concentration of 1.5%.

One of the advantages of such dilution, apart from obtaining exactly the concentrations of phytocompounds required, is to improve the stability and conservation of the plant extract or any mixture of these solvents.

Compositions Comprising a Crocus sativus Flower Extract

A second aspect of the invention is an oral hygiene or therapeutic composition comprising, as active ingredient, an effective amount of Crocus sativus flower extract, and a physiologically acceptable medium.

In an advantageous embodiment, the effective amount of the Crocus sativus flower extract of the invention corresponds to a concentration of between 0.5% and 10%, preferably to a concentration of between 0.8% and 2%, and even more preferably to a concentration of 1%.

The composition that can be used according to the invention can be applied by any suitable route, in particular orally or topically externally, and the formulation of the compositions can be adapted by a person skilled in the art.

Preferably, the compositions according to the invention are in a form suitable for topical application, which may be in a more or less fluid form, such as a mouthwash or a gel. These compositions must therefore contain a physiologically acceptable medium with a suitable pH, i.e. compatible with the oral mucosa, the skin and the appendages, without any risk of discomfort during application, and cover all suitable cosmetic forms.

Examples of physiologically acceptable media commonly used in the field of application under consideration include adjuvants required for formulation, such as solvents, thickeners, diluents, antioxidants, preservatives, perfumes, odour absorbers, etc.

In all cases, a person skilled in the art will ensure that these adjuvants and their proportions are chosen in such a way as not to impair the advantageous properties sought in the composition according to the invention. These adjuvants may, for example, correspond to 0.1 to 25% of the total weight of the composition. When the composition according to the invention is a gel, the thickening agent may represent from 10 to 25% by weight and preferably from 20 to 25% by weight relative to the total weight of the composition.

Uses of Compositions Containing Crocus sativus Flower Extract

The invention thirdly relates to the non-therapeutic use of the composition for oral hygiene.

This particular embodiment of the invention concerns a subject with healthy oral mucosa who wishes to apply care to prevent the appearance of pathologies such as periodontitis or gingivitis or to protect the oral cavity and in particular the gums.

A fourth aspect of the invention is a composition comprising the Crocus sativus flower extract of the invention for its therapeutic use in the prevention or treatment of disorders of the oral cavity.

In this embodiment of the invention, the application is preferably topical.

In a particular embodiment, the oral cavity conditions concerned are bleeding, inflammation, gingival recession, tooth loosening or loss of conjunctival substance associated with periodontitis and gingivitis.

The purpose of this invention is to provide a new active ingredient capable of targeting the activity of the enzyme gingipain, in order to specifically limit the activity of the Porphyromonas gingivalis bacterium while preserving the oral microbiome.

In a particular embodiment, the diseases of the oral cavity concerned are periodontitis and/or gingivitis, when these diseases are caused by the bacterium Porphyromonas gingivalis.

The specificity of the antibacterial potential of a composition comprising the Crocus sativus flower extract of the invention, up to a concentration of 10% (v/v), was tested using the commensal bacteria Streptococcus mutans and Streptococcus gordoni. No growth inhibition of these two bacterial strains was observed.

The specificity of the inhibition of gingipain activity was assessed by measuring the collagenase activity of gingipain in the presence of the 1% Crocus sativus extract of the invention. No reduction in collagenase activity was observed.

The Crocus sativus flower extract of the invention was tested on certain biological markers associated with the virulence of Porphyromonas gingivalis bacteria, cell lysis mechanisms and the inflammatory response.

The Crocus sativus flower extract of the invention has demonstrated its effectiveness in inhibiting the activity of gingipains secreted by Porphyromonas gingivalis, and more particularly arginine-specific gingipains.

The invention also relates to a composition comprising a Crocus sativus flower extract of the invention for its therapeutic use, in which the gingipain enzymes, and more particularly the arginine-specific gingipain enzymes, are inhibited.

Inhibiting the activity of gingipain enzymes on gingival tissue limits the destruction of the gingival epithelium and conjunctival matrix.

Crocus sativus flower extract has also been shown to be effective in preventing red blood cell lysis.

Crocus sativus flower extract has also been shown to be effective in orienting and increasing the anti-inflammatory response.

The Crocus sativus flower extract of the invention also has an anti-bleeding action, demonstrated by anti-haemolytic activity on human red blood cells.

The Crocus sativus flower extract of the invention made it possible to increase the recruitment of macrophages involved in the anti-inflammatory response, particularly M2-type macrophages.

### EXAMPLES

By way of illustration, examples of how the process according to the invention can be carried out are described below.

### Example 1: Preparation of a Crocus sativus Flower Extract

### Extraction Process

In a first step a), 25 g of dried pistil-free Crocus sativus flowers are ground and then mixed with 975 g of a mixture of distilled water and propanediol at a ratio of 70/30%, i.e. 2.5% of the raw material used in the process and 97.5% of the water/propanediol mixture for a total weight of 1 kg.

b) The mixture is gently stirred for one hour at a temperature of 45° C. to allow the maceration process to take place.

c) The mixture obtained in b) is then purified by centrifugation at 4000 g for 10 min, so as to sediment the residual plant material in the pellet and collect the supernatant.

d) The supernatant obtained in the previous step is clarified by sequential filtration steps involving at least six filtrations with decreasing porosity ranging from 30 to 0.2 μm.

e) A final sterilising filtration stage is carried out at 0.22 μm.

f) The pH is measured but does not need to be adjusted.

The Crocus sativus flower extract thus obtained is characterised by an amber to dark amber colour, with a slightly viscous and opalescent consistency, a density of 1.03 g/cm³ and a pH of 5.7. This extract has been analysed by standard methods and contains a quantity of dry matter of 12 g/Kg, a sugar content of 8.7 g/kg, amino acids of 0.55 g/kg, phenolic compounds of 0.85 g/kg and proteins of 4.5 g/kg.

Characterisation of the Crocus sativus Flower Extract

The analyses were carried out on several extracts in order to establish a concentration range for each molecule analysed.

### Methods Used:

The overall composition of the extract is shown in Table 1.

The dry weight of the extract was measured after evaporation (12 hours drying at 105° C.).

The total sugar content was measured by the following method: dissolving the extract in concentrated sulphuric acid and then reacting with phenol to form a coloured complex. The absorbance of the complex was measured spectrophotometrically at 490 nm. The sugar content was determined using a standard glucose curve.

The free amino acid content of the extract was assessed by the following method: breaking down the amine and carboxyl functions with the ninhydrin reagent. The absorbance of the coloured complex was measured spectrophotometrically at 570 nm. Quantification was determined using a standard curve based on a pool of amino acids.

The total content of phenolic compounds was assessed by the following method: reduction of the extract by the Folin-Ciocalteu reagent and spectophotometric measurement at 760 nm. Phenolic molecules were quantified using a standard gallic acid curve.

Protein content was assessed by the following method: colorimetric reaction using Biuret reagent combined with Folin and Ciocalteu reagent and spectrophotometric measurement at 550 nm. Quantification was carried out using a BSA (Bovine Serum Albumin) standard curve.

TABLE 1

| | Crocus sativus flower extract |
|---|---|
| Dry weight (g/kg) | 9-13 |
| Total sugars (g/kg) | 5.5-8.5 |
| Free amino acids (mg/kg) | 350-550 |
| Total phenolic compounds (g/kg) | 0.4-1.2 |
| Proteins (g/kg) | 2.5-5.5 |
| Vitamins (mg/kg) | 1.2-3.4 |

Identification and detailed quantification of the sugars present was carried out using high-performance liquid chromatography (HPLC), coupled to an ACQUITY Qda mass spectrometer (WATERS) equipped with a negative-mode electrospray probe. Samples were separated on a Luna Omega SUGAR 100A 3 μm (150×4.6 mm) column (Phenomenex: H21-180323) by an Agilent 1260 HPLC system (Agilent Technologies). The flow rate was 0.8 ml/min. The mobile phases consisted of an aqueous solution of 20 mmol ammonium acetate and 95/5 (ACN/H20) 20 mmol ammonium acetate.

TABLE 2

| | | | Glucose |
|---|---|---|---|
| | Xylitol (mg/Kg) | Fructose (mg/Kg) | (mg/Kg) |
| Crocus sativus | <LD-20 | 180-540 | 2800-4800 |
| flower | | | |
| extract | | | |

Identification and detailed quantification of the amino acids present in the extract was carried out by liquid chromatography coupled to a UV detector set at 254 nm wavelength. Samples were separated on an Uptisphere Strategy C18-2 5 μm (250×4.6 mm) column US5C182-250/046 (Interchim: UE2.6AQ-100/046). The flow rate was 1 ml/min. The mobile phases consisted of a 0.1% solution of phosphoric acid (H3PO4) and acetonitrile. Samples and standards were derivatised with phenylisothiocyanate.

TABLE 3

| | | | | | Glutamic | | | | |
|---|---|---|---|---|---|---|---|---|---|
| | Histidine | Arginine | Serine | Glycine | acid | Threonine | Proline | Alanine | Valine |
| | (ppm) | (ppm) | (ppm) | (ppm) | (ppm) | (ppm) | (ppm) | (ppm) | (ppm) |
| Extract | 25-75 | 5-20 | <LD-100 | <LD-40 | 20-60 | 10-40 | 70-190 | 15-45 | 10-30 |

The content of glycosylated flavonoids was determined by high-performance liquid chromatography coupled to a UV detector fixed at 254 nm. Samples were separated on a 100 mm×4.6 mm×2.6 μm Uptisphere CS evolution column (Interchim, UE2-6AQ-100046) using an Agilent 1200 system (Agilent technologies). The flow rate was 0.8 mL/min. The mobile phases consisted of a solution of 0.1% trifluoroacetic acid (TFA) and methanol.

TABLE 4

| | Kaempferol | | |
|---|---|---|---|
| | sophoroside | Kaempferol glucoside | Quercetin diglucoside |
| | (mg/kg) | (mg/kg) | (mg/kg) |
| Extract | 150-450 | 5-30 | 10-40 |

Characterisation and quantification of the organic acids was carried out by high-performance liquid chromatographic analysis, coupled to an ACQUITY Qda mass spectrometer (WATERS) equipped with an electrospray ion source in negative mode. Samples were separated on an EC 150/4.6 Nucleoshell RP 18plus-5 μm column (150×4.6 mm) (Macherey Nagel: 763236.46) by an Agilent 1260 HPLC system (Agilent Technologies). The flow rate was 0.3 ml/min. The mobile phases consisted of a 0.01% solution of formic acid (HCOOH) and acetonitrile.

TABLE 5

| | Citric | Lactic | Succinic | Malic | Uronic |
|---|---|---|---|---|---|
| | acid | acid | acid | acid | acid |
| | (mg/Kg) | (mg/kg) | (mg/kg) | (mg/kg) | (mg/kg) |
| Extract | 20-80 | <LD-10 | 30-90 | 200-600 | 50-150 |

Vitamins were characterised and quantified using high-performance liquid chromatographic analysis, coupled to an ACQUITY Qda mass spectrometer (WATERS) equipped with a positive mode electrospray ion source. Samples were separated on an EC 150/4.6 Nucleoshell RP 18plus-5 μm column (150×4.6 mm) (Macherey Nagel: 763236.46) by an Agilent 1260 HPLC system (Agilent Technologies). The flow rate was 0.5 ml/min. The mobile phases consisted of an aqueous solution of 20 mmol ammonium acetate, 0.1% formic acid and a 65/35 methanol/acetonitrile solution of 20 mmol ammonium acetate, 0.1% formic acid.

TABLE 6

| | | | B3-b- | |
|---|---|---|---|---|
| | B2- | B3-a- | Nicotinic | |
| | Riboflavin | Nicotinamide | acid | B5-Pantothenic |
| | (mg/kg) | (mg/kg) | (mg/kg) | acid (mg/kg) |
| Extract | 0.06-0.18 | 0.3-0.9 | 0.2-0.6 | 0.5-1.5 |

### Example 2: Evaluation of the Sativus Flower Extract from Example 1 on the Activity of the Gingipain Enzymes KgP and RgP

Principle: Gingipain enzymes are one of the virulence factors of the Porphyromonas gingivalis bacterium. Gingipains have the ability to digest a broad spectrum of host proteins. There are lysine-specific (KgP) and arginine-specific (RgP) gingipain enzymes.

Protocol: Porphyromonas gingivalis bacteria were cultured in Shaedler broth supplemented with vitamin K for forty-eight hours. The broth containing the bacteria was then centrifuged at 10,000×g for 5 minutes at 4° C. and the bacterial pellet was rinsed twice with 1×PBS. The bacteria were then transferred to 1×PBS to obtain an aliquot with an optical density (OD) of 1 and an aliquot with OD=2. The OD=1 and OD=2 aliquots were incubated at 37° C. for 1 hour and then centrifuged at 10,000×g for 10 minutes at room temperature. The supernatant was mixed with a chromogenic substrate specific for RgP or KgP gingipain activity (benzoyl-arginine-p-nitroanilide (TGPpNA) or benzoyl-lysine-p-nitroanilide (BAPNA) respectively). Hydrolysis of the arginine or lysine bond releases the p-nitroaniline chromophore, which can be quantified colorimetrically.

The measurements were carried out in the presence or absence of the Crocus sativus flower extract from example 1 at 1% final in water (volume/volume) or in the presence of a reference inhibitor; iodoacetamide at 10 mM.

The release of the chromophore p-nitroaniline was quantified every 15 minutes for 2 hours by spectrophotometry at 405 nm. The activity of gingipain was calculated as a percentage of the maximum OD obtained for the RgP control (CTR) or the KgP control (CTR) at 2 hours.

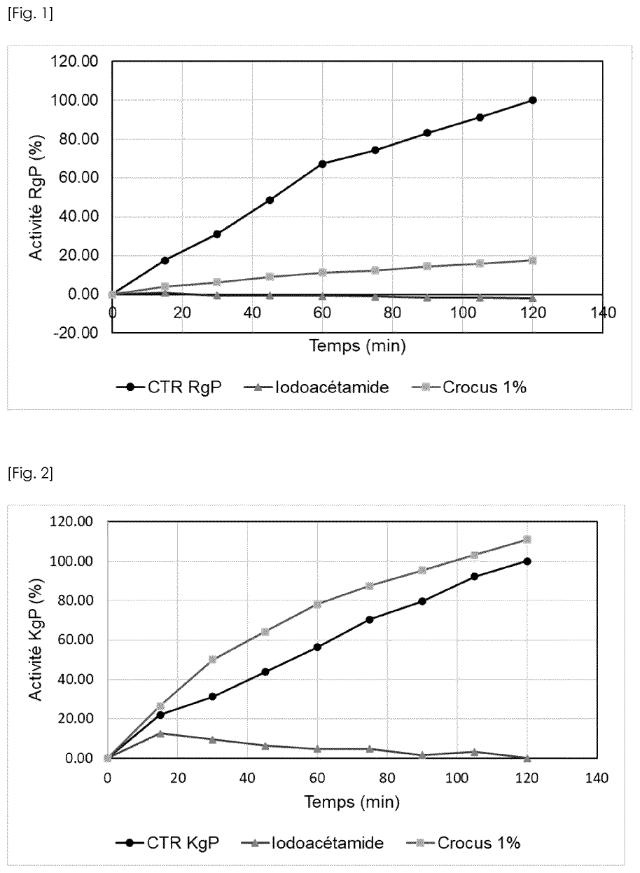

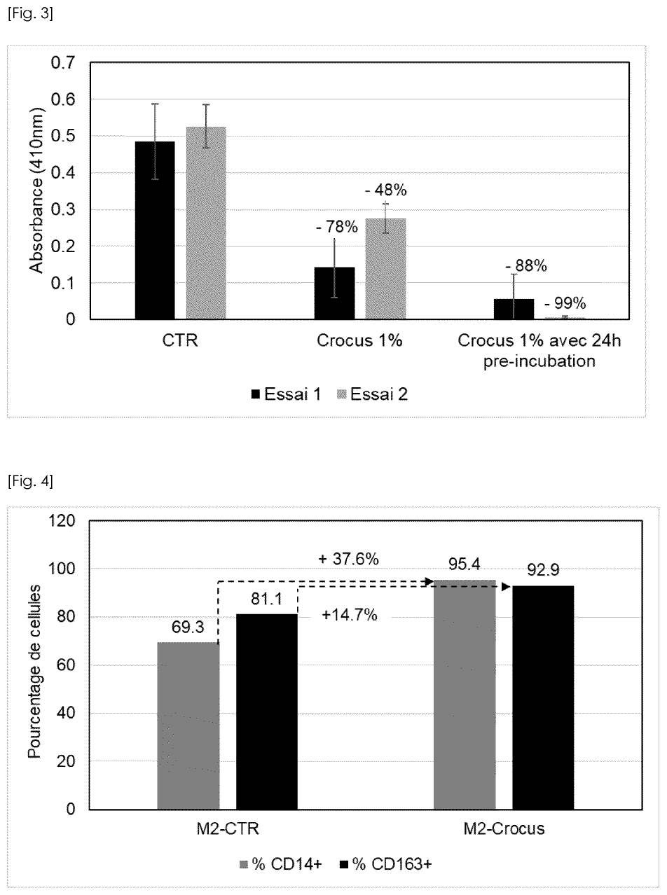

Results: In the presence of the 1% Crocus sativus flower extract from example 1, we observed a decrease in enzymatic activity of up to 85.5% compared to the CTR RgP for the 2 h time point, as illustrated in [FIG. 1]. In contrast, there was no decrease in activity compared with CTR KgP in the presence of 1% Crocus sativus flower extract ([FIG. 2]).

Conclusion: 1% Crocus sativus flower extract inhibits arginine-specific gingipains and has no effect on lysine-specific gingipains. By inhibiting the activity of gingipains, Porphyromonas gingivalis bacteria no longer have the tools they need to lyse cell junctions and damage extracellular matrix proteins. As gingipains are one of the most important virulence factors of P. gingivalis, their inhibition limits the development of these bacteria and thus prevents the onset of gingivitis and periodontitis.

### Example 3: Evaluation of the Anti-Haemolytic Activity of the Sativus Flower Extract from Example 1 on Human Red Blood Cells

Principle: Certain pathogens secrete enzymes which are capable of lysing the membrane of red blood cells (haemocytes), thereby releasing haemoglobin into the environment: this is haemolytic activity.

Protocol: Fresh human blood stored at room temperature is centrifuged at 1500×g for 10 minutes, the supernatant is removed and the red blood cells contained in the pellet are recovered with 50 ml of a 0.05% BSA-PBS solution. The medium was shaken by inverting. The operation was repeated 3 times to wash the cells thoroughly and eliminate extracellular haemoglobin. After the final rinse, as much of the supernatant as possible was removed and the pellet was mixed with the PBS-0.05% BSA solution to obtain a 1% red cell solution. Porphyromonas Gingivalis bacteria are cultured in Shaedler broth supplemented with vitamin K for 24 to 48 hours, in the presence or absence of the 1% Crocus sativus flower extract from example 1. The broths containing the bacteria were then centrifuged at 10,000×g for 5 minutes at 4° C. The bacteria are recovered in 1×PBS to obtain an aliquot with an OD of between 0.5 and 1.0 UDO. In a well of a 96-well plate, 100 μL of bacterial suspension is brought into contact with 100 ul of 1% red cell solution and 50 μL of Crocus sativus extract at the concentration tested (1% in PBS volume/volume) or 50 μL of PBS for control conditions. This mixture was then shaken at 37° C. for 24 hours, followed by centrifugation at 1500×g. The absorbance of the supernatant was read with a microplate reader at a wavelength of 410 nm. Samples were plated in five replicates per assay, with two independent assays (assay 1 and assay 2).

Result: Application of the 1% Crocus sativus flower extract of example 1 induces a reduction in haemolysis of between 48 and 78% compared to the negative control after incubation for 24 hours. When P. gingivalis bacteria are pre-incubated with the 1% Crocus sativus flower extract for 24 hours, the reduction in haemolysis is between 88 and 99%. The results are illustrated in [FIG. 3].

Haemolysis of red blood cells releases haemoglobin rich in haem groups, which the P. gingivalis bacterium needs to develop. By inhibiting haemolysis, P. gingivalis is deprived of haem, which slows down the development of this bacterium and prevents the gum bleeding seen in gingivitis and periodontitis.

### Example 4 Evaluation of the Sativus Flower Extract from Example 1 for the Orientation of the Inflammatory Response Involving PBMC-Derived Macrophages

Principle: the aim of this experiment is to test an active ingredient to determine whether this extract has the potential for orientation of the immune response. By orientation we mean the ability of the extract to promote a pro-inflammatory response (involving M1-type macrophages) or an anti-inflammatory response (involving M2-type macrophages). This orientation is measured by analysing the cluster present on the surface of macrophage cells specific to the inflammatory response using a flow cytometer. A macrophage population with few CD14 and CD163 clusters is specific to an M1 type response, while a population rich in CD14 and CD163 is specific to an M2 type response.

Protocol: Specialised PBMC (peripheral blood mononuclear cells) are isolated from fresh blood by Ficoll gradient centrifugation. After centrifugation, the PBMC cells are recovered by aspiration of a cell ring present between the different phases. CD14 cells were isolated by positive selection on CD14-labelled magnetic beads. After isolation, the cells were rinsed with a cold PBS/BSA/EDTA solution and this was repeated several times. The cells thus obtained are seeded in culture dishes or flasks containing a specific attachment medium. After 2 to 3 hours of incubation, the medium is aspirated and replaced by a culture medium that promotes the proliferation and differentiation of PBMC into M1 and M2 macrophage cells. Specific differentiation of cells into M1 macrophage type is achieved by adding GMCSF to the medium and into M2 macrophage type with MCSF for 24 hours. Crocus sativus flower extract from example 1 or PBS control at a final concentration of 1% (v/v) is added 24 hours before the start of terminal cell differentiation. The cells are then detached from their support after trypsin action for analysis by flow cytometry.

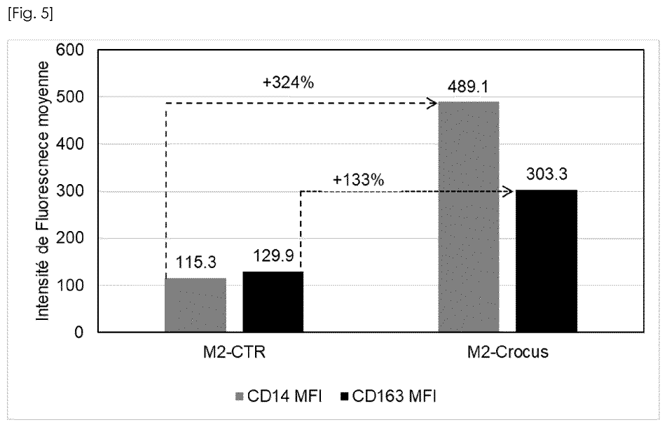

Results: Application of the 1% Crocus sativus flower extract from example 1 to the macrophage culture medium for 24 hours induced an increase in the M2-type anti-inflammatory response, resulting in an increase in the number of cells displaying the CD14 cluster on its surface (from 69.3% to 95.9% of the total population, i.e. an increase of 37.6%) as shown in [FIG. 4] and an increase in MFI (from 115.3 to 489.1, an increase of 324.2%) as shown in [FIG. 5]. In addition, the CD163 cluster, which specialises in the anti-inflammatory response, was also increased (from 81.1% to 92.9%, i.e. an increase of 14.7%) as was its MFI (from 129.9% to 303.3%, i.e. an increase of 134.2%).

The above results indicate that 1% Crocus sativus flower extract regulates the immune response, thereby promoting an anti-inflammatory response.

### Example 5 Formulation of a Mouthwash Containing Crocus sativus Flower Extract

TABLE 7

| Ingredients | % (v/v) |
|---|---|
| Crocus sativus flower extract from example 1 | 1.0 |
| 70% sorbitol | 10 |
| Glycerine | 10 |
| Propylene glycol | 5.0 |
| Xylitol | 2.0 |
| Erythritol | 1.0 |
| Sodium cocamidopropyl betaine | 0.30 |
| Natural peppermint aroma | 0.1 |
| Benzyl alcohol | 0.50 |
| Raspberry Cetone | 0.50 |
| water | q.s.p 100 ml |

### Example 6 Formulation of a Gel Containing Crocus sativus Flower Extract

TABLE 8

| Ingredients | % (v/v) |
|---|---|
| Crocus sativus flower extract from example 1 | 1.0 |
| NatraThix ™ Bio cellulose | 1.2 |
| Glycerine | 25 |
| Sorbitol solution (70%) | 20 |
| Propylene glycol | 5.0 |
| Xylitol | 2.0 |
| Erythritol | 1.0 |
| Menthol | 0.40 |
| Benzyl alcohol | 0.50 |
| Raspberry Cetone | 0.50 |
| Water | q.s.p 100 |

What is claimed is:

1. A Crocus sativus flower extract comprising from 9 to 13 g/Kg of dry matter, from 5.5 to 8.5 g/kg of total sugars, from 2.5 to 5.5 g/kg of proteins, from 0.35 to 0.55 g/Kg of amino acids, from 0.6 to 1.2 g/kg of organic acids, from 0.4 to 12 g/kg of total phenolic compounds including 150 to 450 mg/kg of kaempferol sophoroside, from 5 to 30 mg/kg of kaempferol glucoside and from 10 to 40 mg/kg of quercetin diglucoside, wherein said extract is obtained by the process comprising the following steps:
a) fresh or dried flowers without pistils are placed in the presence of propanediol at a ratio of 30%, as a hydro-polyalcoholic solvent,
b) the mixture is gently stirred for 30 minutes to two hours at a temperature of between room temperature and 80° C.,
c) the mixture obtained in b) is purified to eliminate the solid vegetable matter and recover the liquid part,
d) the extract obtained in the previous step is clarified by at least six sequential filtration steps using filters with decreasing porosities ranging from 30 to 0.2 μm,
e) sterilising filtration is carried out using a 0.22 μm porosity filter.

2. The Crocus sativus flower extract according to claim 1, wherein in step a) the flowers are dried and ground.

3. The Crocus sativus flower extract according to claim 1, wherein in step b), the mixture is gently stirred for 30 minutes to one and a half hours at a temperature of between 30° C. and 50° C.

4. An oral hygiene or therapeutic composition comprising, as active ingredient, an effective amount of the Crocus sativus flower extract according to claim 1, and a physiologically acceptable medium.

5. A composition according to claim 4, wherein the effective amount of Crocus sativus flower extract corresponds to a concentration of between 0.5% and 10%, preferably to a concentration of between 0.8% and 2%, and even more preferably to a concentration of 1%.

6. A method of non-therapeutic treatment for oral hygiene comprising applying the composition according to claim 4 to a healthy oral cavity.

7. A method of therapeutic treatment, comprising applying the composition according to claim 4 to oral cavity for the prevention and treatment of diseases of the oral cavity.

8. A method of therapeutic treatment, comprising applying the composition according to claim 4 to oral cavity for the prevention and treatment of diseases of the oral cavity wherein the diseases of the oral cavity are bleeding, inflammation, gingival recession, dental loosening or loss of conjunctival substance associated with periodontitis and gingivitis.

9. A method of therapeutic treatment, comprising applying the composition according to claim 4 to oral cavity, for the prevention and treatment of diseases of the oral cavity wherein the diseases of the oral cavity are caused by the bacterium Porphyromonas gingivalis.

10. A method of therapeutic treatment, according to claim 9, wherein the activity of the gingipain enzymes secreted by the bacterium Porphyromonas gingivalis is inhibited.

11. A method of therapeutic treatment according to claim 10, wherein the gingipain enzymes are arginine-specific gingipains.
